(19) United States
(12) Patent Application Publication
(10) Pub. No.: US 2019/0153021 A1
(43) Pub. Date: May 23, 2019

(54) THERAPEUTIC COMPOUNDS

(71) Applicant: UNIVERSITY OF IOWA RESEARCH FOUNDATION, Iowa City, IA (US)

(72) Inventors: Zhendong Jin, Iowa City, IA (US); Yan Mei, Iowa City, IA (US); Lei Chen, Iowa City, IA (US); Aashay Sha, Iowa City, IA (US)

(73) Assignee: UNIVERSITY OF IOWA RESEARCH FOUNDATION, Iowa City, IA (US)

(21) Appl. No.: 15/781,642

(22) PCT Filed: Dec. 6, 2016
(86) PCT No.: PCT/US16/65059

§ 371 Date: Jun. 5, 2018

Related U.S. Application Data
(60) Provisional application No. 62/264,056, filed on Dec. 7, 2015.

(51) Int. Cl. C07J 17/00; A61K 9/20

(52) U.S. Cl. CPC C07J 17/005; A61K 9/2054; A61K 9/2027; A61K 9/2009; A61K 9/2013; A61K 9/2059; A61K 9/2018

(57) ABSTRACT

The invention provides compounds of formula (I): and salts thereof, wherein R¹, R², R³, B, X, Y, and Z have any of the values defined herein. The invention also provides pharmaceutical compositions comprising a compound of formula (I) or a pharmaceutically acceptable salt thereof, processes for preparing compounds of formula (I) and salts thereof, intermediates useful for preparing compounds of formula (I) and salts thereof, and therapeutic methods for treating cancer using a compound of formula (I) or a pharmaceutically acceptable salt thereof.

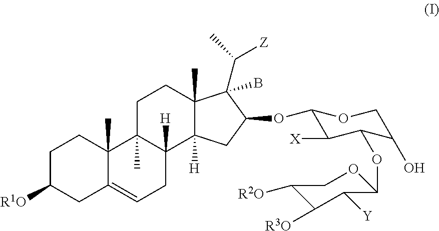

### RELATED APPLICATION

This application claims priority of U.S. Provisional Application Serial No. 62/264,056 filed on Dec. 7, 2015, which application is incorporated by reference herein.

### FIELD OF THE INVENTION

The disclosure describes therapeutic compounds useful in the treatment of diseases.

### BACKGROUND OF THE INVENTION

In the last several decades many natural saponins have been isolated and a number of them exhibit remarkable anticancer activities (Man, S.; et al., Fitoterapia 2010, 81, 703 and references cited therein.). OSW-1 (Scheme 1), isolated from the bulbs of Ornithogalum saundersiae, a perennial grown in southern Africa, is considered as the crown jewel among anticancer saponins due to its extremely potent anticancer activity against a wide spectrum of cancer cells (a mean IC50 of 0.78 nM in the NCI 60-cell in vitro screen) (Mimaki, Y. et al., Bioorganic Med. Chem. Lett. 1997, 7, 633 and references cited therein). It is one of the most potent anticancer agents ever tested at NCI (For the GI50, TGI, and LC50 values of OSW-1 against the NCI 60 cell-line tumor panel, see the Supporting Information of the following reference: Kuroda, M. et al., J. Nat. Prod. 2001, 64, 88.). Relatively, its anticancer activities are about 10-100 times more potent than many well-known anticancer drugs in clinical use. Because the isolation yield of OSW-1 from the plant is only 0.0027%, an efficient synthetic process (28% overall yield) has been developed and has successfully solved the bottleneck of OSW-1 supply ((a) Yu, W.; Jin, Z., J. Am. Chem. Soc. 2001, 123, 3369. (b) Yu, W.; Jin, Z., J. Am. Chem. Soc. 2002, 124, 6576. (c) Zhendong Jin, Wensheng Yu, U.S. Pat. No. 6,753,414. Filing date: Aug. 6, 2002). Biological studies carried out at MD Anderson Cancer Center using OSW-1 showed that unlike other anticancer agents that inhibit DNA synthesis or block cell cycle progression, OSW-1 involves a mitochondria-mediated mechanism that is independent of cell cycle and can effectively kill dormant cancer cells at sub-nM concentrations ((a) Zhou, Y. et al., J. Natl. Cancer Inst. 2005;97:1781-1785. (//dx.doi.org doi: 10.1093/jnci/dji404) (b) Celia Garcia-Prieto et al., J. Biol. Chem. 2013, 288, 3240.). Thus, OSW-1 represents a new promising lead compound for the development of an effective anticancer agent with a novel mechanism of action. However, to advance OSW-1 into clinical evluation it is important to improve its metabolic stability and pharmacokinetics.

### SUMMARY OF THE INVENTION

The present invention provides compounds that act as agents for treating and preventing cancer.

Accordingly the invention provides a compound of formula (I):

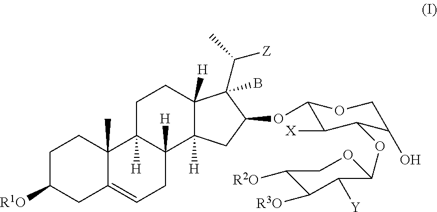

wherein:

R¹ is H, (C₁-C₆)alkyl, (C₁-C₆)alkanoyl, (C₁-C₆)alkoxycarbonyl, -C(═O)NR^{a}R^{b}, or a saccharide;

R² is H, (C₁-C₆)alkyl, (C₁-C₆)alkanoyl, (C₁-C₆)alkoxycarbonyl, or —C(═O)NR^{a}R^{b};

R³ is H, (C₁-C₆)alkyl, (C₁-C₆)alkanoyl, (C₁-C₆)alkoxycarbonyl, or —C(═O)NR^{a}R^{b};

B is H, hydroxy, NH₂, or (C₁-C₆)alkyl;

X is hydroxy, (C₁-C₆)alkoxy, (C₁-C₆)alkanoyloxy, (C₁-C₆)alkanoyloxycarbonyl, —O—C(═O)NR^{a}R^{b}, —NH—(C═O)OR^{c}, —NH—(C═O)R^{e}R^{d}, —NH—S(═O)₂R^{c}, —NH—(S═O)R^{c}, —O-saccharide, cinnamoyl, or —NR^{c}R^{d};

Y is hydroxy, (C₁-C₆)alkoxy, (C₁-C₆)alkanoyloxy, (C₁-C₆)alkanoyloxycarbonyl, —O—C(═O)NR^{a}R^{b}, —NH—(C═O)OR^{c}, —NH—(C═O)NR^{c}R^{d}, —NH—S(═O)₂R^{c}, —NH—(S═O)R^{c}, —O-saccharide, cinnamoyl, or —NR^{c}R^{d};

Z is H, hydroxy, (C₁-C₈)alkyl, (C₁-C₆)alkoxycarbonyl, (C₁-C₆)alkanoyloxy, (C₁-C₆)alkylthiocarbonyl, (C₁-C₆)alkanoylthio, (C₂-C₈)alkenyl, -C(═O)NR^{a}R^{b}, —NR^{a}R^{b}, or (C₂-C₈)alkynyl, wherein any (C₁-C₈)alkyl, (C₁-C₆)alkoxycarbonyl, (C₁-C₆)alkanoyloxy, (C₁-C₆)alkylthiocarbonyl, (C₁-C₆)alkanoylthio, (C₂-C₈)alkenyl, and (C₂-C₈)alkynyl, is optionally substituted with one or more groups independently selected from halo and (C₁-C₆)alkoxy; each R^{a} and R^{b} is independently H, (C₁-C₆)alkyl, aryl, heteroaryl, aryl(C₁-C₆)alkyl or heteroaryl(C₁-C₆)alkyl, wherein any (C₁-C₆)alkyl, aryl, heteroaryl, aryl(C₁-C₆)alkyl and heteroaryl(C₁-C₆)alkyl of R^{a} and R^{b} is optionally substituted with one or more groups independently selected from halo, cyano, oxo (═O), —S(O)—R^{e}, (C₁-C₆)alkyl, (C₃-C₆)cycloalkyl, carboxy, NO₂, hydroxy, (C₁-C₆)alkoxy, aryl, and heteroaryl; or R^{a}and R^{b} together with the nitrogen to which they are attached form a morpholino, piperazino, pyrrolidino or piperidino ring, wherein the morpholino, piperazino, pyrrolidino and piperidino ring is optionally substituted with one or more groups independently selected from halo, cyano, oxo (—O), (C₁-C₆)alkyl, (C₃-C₆)cycloalkyl, carboxy, NO₂, hydroxy, (C₁-C₆)alkoxy, (C₁-C₆)alkoxycarbonyl, and (C₁-C₆)alkanoyloxy;

each R^{c} and R^{d} is independently H, (C₁-C₆)alkyl, aryl, heteroaryl, aryl(C₁-C₆)alkyl or heteroaryl(C₁-C₆)alkyl, wherein any (C₁-C₆)alkyl, aryl, heteroaryl, aryl(C₁-C₆)alkyl and heteroaryl(C₁-C₆)alkyl of R^{a} and R^{b} is optionally substituted with one or more groups independently selected from halo, cyano, oxo (—O), (C₁-C₆)alkyl, (C₃-C₆)cycloalkyl, carboxy, NO₂, hydroxy, (C₁-C₆)alkoxy, aryl, and heteroaryl; or R^{e} and R^{d} together with the nitrogen to which they are attached form a morpholino, piperazino, pyrrolidino or piperidino ring, wherein the morpholino, piperazino, pyrrolidino and piperidino ring is optionally substituted with one or more groups independently selected from halo, cyano, oxo (—O), (C₁-C₆)alkyl, (C₃-C₆)cycloalkyl, carboxy, NO₂, hydroxy, (C₁-C₆)alkoxy, (C₁-C₆)alkoxycarbonyl, and (C₁-C₆)alkanoyloxy;

each R^{e} is independently H, (C₁-C₆)alkyl, aryl, heteroaryl, aryl(C₁-C₆)alkyl or heteroaryl(C₁-C₆)alkyl, wherein any (C₁-C₆)alkyl, aryl, heteroaryl, aryl(C₁-C₆)alkyl and heteroaryl(C₁-C₆)alkyl of R^{a} and R^{b} is optionally substituted with one or more groups independently selected from halo, cyano, oxo (—O), (C₁-C₆)alkyl, (C₃-C₆)cycloalkyl, carboxy, NO₂, hydroxy, (C₁-C₆)alkoxy, aryl, and heteroaryl; and

each n is independently 0, 1, or 2;

or a salt thereof.

The invention also provides a composition comprising a compound of formula (I), or a pharmaceutically acceptable salt thereof, and a pharmaceutically acceptable diluent or carrier.

The invention also provides a method for treating or preventing cancer in an animal comprising administering a compound of formula (I) or a pharmaceutically acceptable salt thereof to the animal.

The invention also provides a compound of formula (I) or a pharmaceutically acceptable salt thereof for use in medical therapy.

The invention also provides a compound of formula (I) or a pharmaceutically acceptable salt thereof for the prophylactic or therapeutic treatment of cancer.

The invention also provides a compound of formula (I) or a pharmaceutically acceptable salt thereof to prepare a medicament for treatment of cancer in an animal.

### DETAILED DESCRIPTION

It was discovered that the relatively weak in vivo efficacy of OSW-1 is due to its metabolic instability and poor pharmacokinetics (PK) (Z. Jin, S. Chen, and D. Murry unpublished results.); the two ester groups of OSW-1 are quickly cleaved by enzymes in mouse blood to give a major metabolite 2 that is 1,000 times less active (Scheme 1).

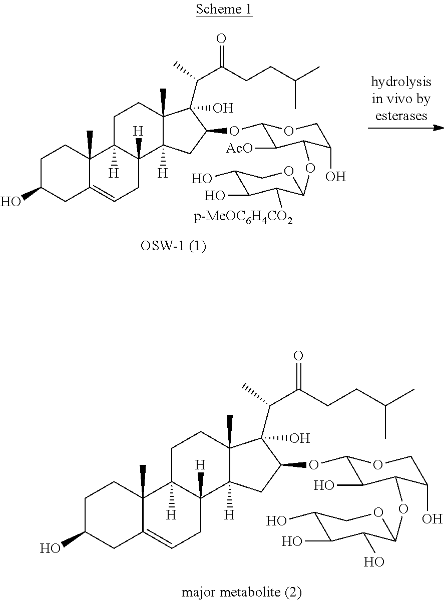

It was reported that compound 3 with a simplified steroidal aglycone showed similar in vitro anticancer activity to compound 1 (Scheme 2) (Deng, L. H. et al., Bioorg. Med. Chem. Lett. 2004, 14, 2781). Therefore, it was decided to use this simplified steroidal aglycone when designing novel compound 4.

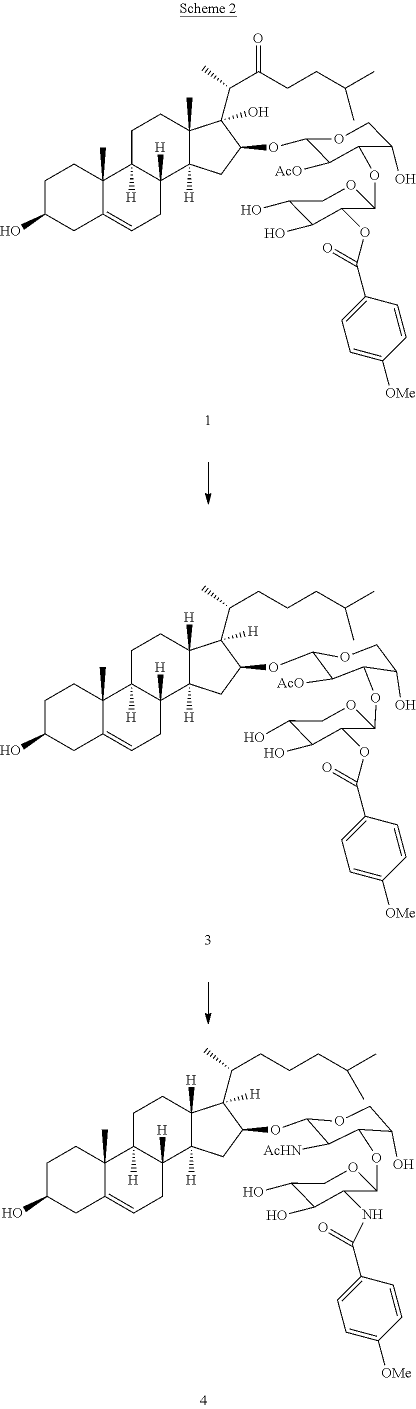

### Retrosynthetic Analysis

The retrosynthetic analysis of compound 4 is outlined in Scheme 3. Sequential disconnections at the glycoside bonds revealed the aglycone 7 and two monosaccharide units 8 and 6. Both monosaccharide units 8 and 6 can be derived from L-arabinose 10 and D-xylose 9, respectively.

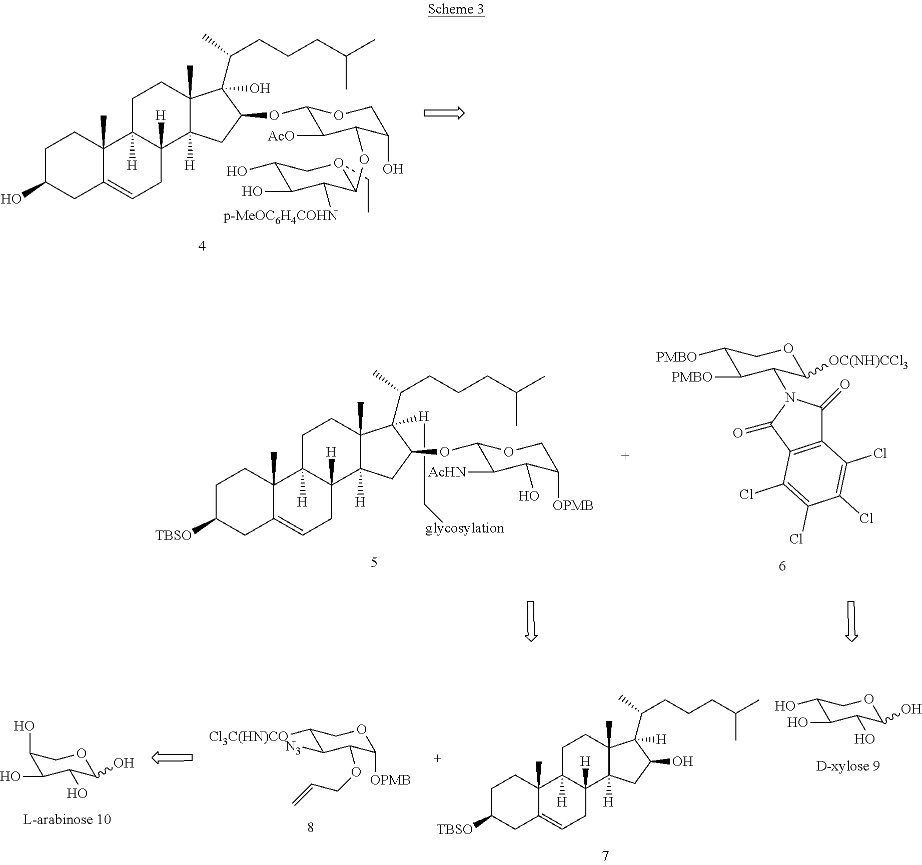

### Synthesis of Aglycone 7

Commercially available diosgenin 11 was reduced using a modified Clemmensen reduction in the presence of Zn dust and 19% HCl to obtain the triol 12 with 73% yield (Martin, R. et al. Organic & Biomolecular chemistry 2009, 7, 909.). The triol 12 reacted with excess triethylsilane and frustrated Lewis acid B(C₆F₅)₃ in CH₂Cl₂for 6 days to undergo a modified Barton-McCombie deoxygenation reaction to obtain the TES protected steroid 13 (Denancé, M et al., Steroids 2006, 71, 599.). The reaction mixture was run through a short silica-gel column to remove the baseline impurities and was exposed to a catalytic amount of TsOH to cleave the two silyl protecting groups to afford the diol 14 with an overall yield of 63% for 2 steps. Finally the diol 14 was regioselectively protected with a TBS protecting group at the C-3 position to afford desired steroidal aglycone 7 with an 89% yield.

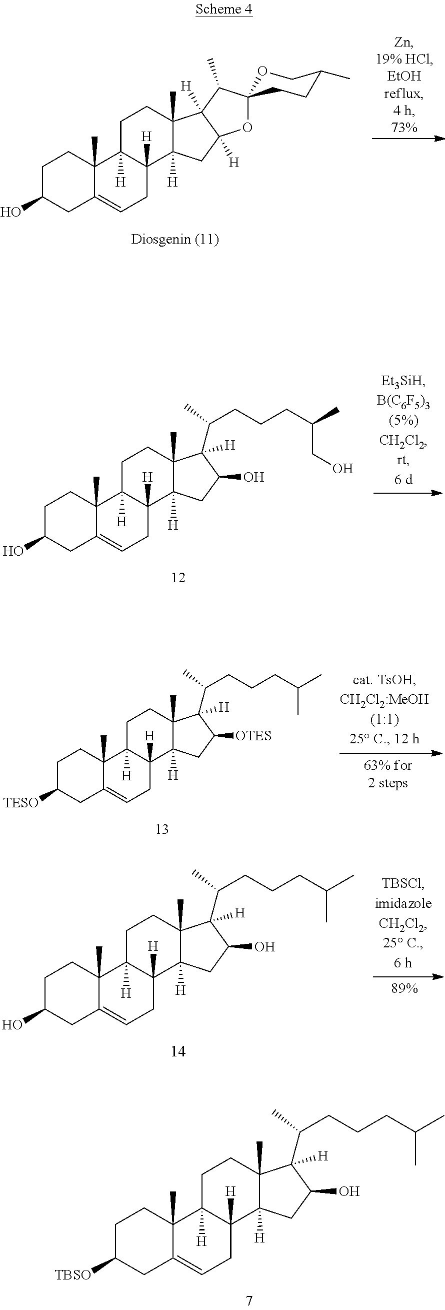

### Synthesis of Monosaccharide Unit 8

The synthesis of monosaccharide unit 8 is shown in Scheme 5. L-arabinose 10 was converted to compound 16 employing a three-step reaction sequence (Balog, A. et al., Syn. Comm. 1996, 26, 935-944.). Compound 16 reacted with NaN₃ in the presence of CAN to provide compound 17 (Hashimoto, H. et al., J. Bull. Chem. Soc. Jpn. 1986, 59, 3131.). The nitrate group at the anomeric position of compound 17 was cleaved by thiophenol to give compound 18. TIPS protection followed by hydrolysis afforded compound 20 in excellent yield. Regioselective allyl protection of the C-3 alcohol followed by protecting the C-4 alcohol with a PMB group gave compound 22. Removal of the TIPS group followed by the reaction with trichloroactonitrile in the presence of a catalytic amount of DBU furnished compound 8 (Schmidt, R. R.; Michel, J. Tetrahedron Lett. 1984, 25, 821.)

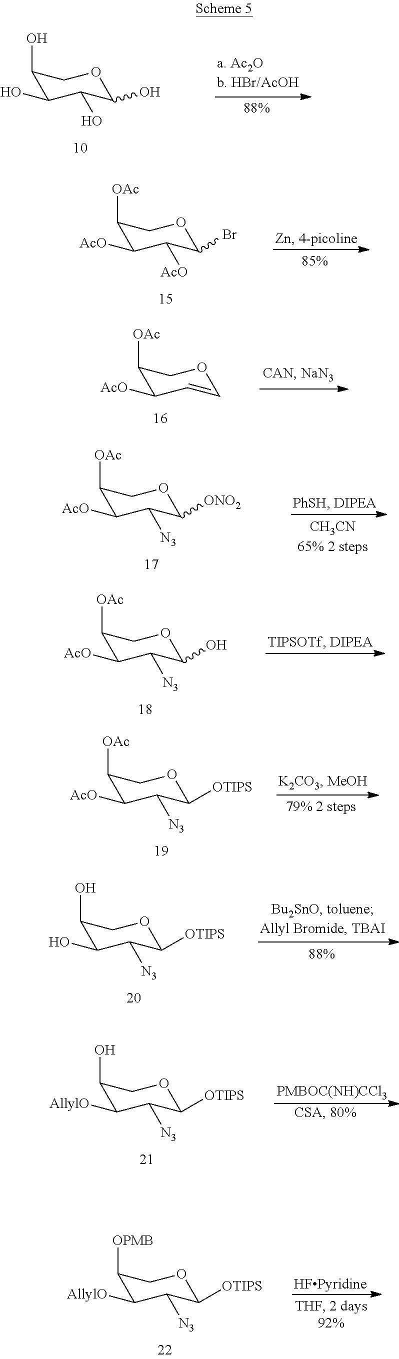

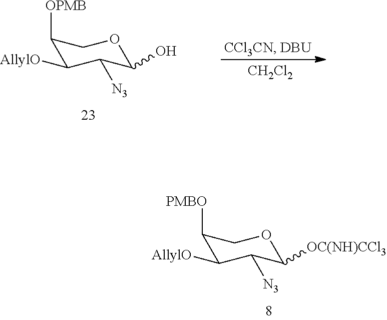

### Synthesis of Monosaccharide Unit 6

Monosaccharide unit 6 was prepared from D-xylose 9 using a similar strategy (Scheme 6). D-xylose 9 was converted to compound 25 employing a three-step reaction sequence. Compound 25 reacted with NaN₃ in the presence of CAN to provide compound 26. The nitrate group at the anomeric position of compound 26 was cleaved by thiophenol to give compound 27. TIPS protection followed by hydrolysis afforded compound 29 in excellent yield. Protection of both C-3 and C-4 alcohols with PMB groups gave compound 30. Reduction of azide to amine followed by the formation of tetrachlorophthalimide provided compound 31. Removal of the TIPS group followed by the reaction with trichloroactonitrile in the presence of a catalytic amount of DBU furnished compound 6 (Schmidt, R. R.; Michel, J. Tetrahedron Lett. 1984, 25, 821.).

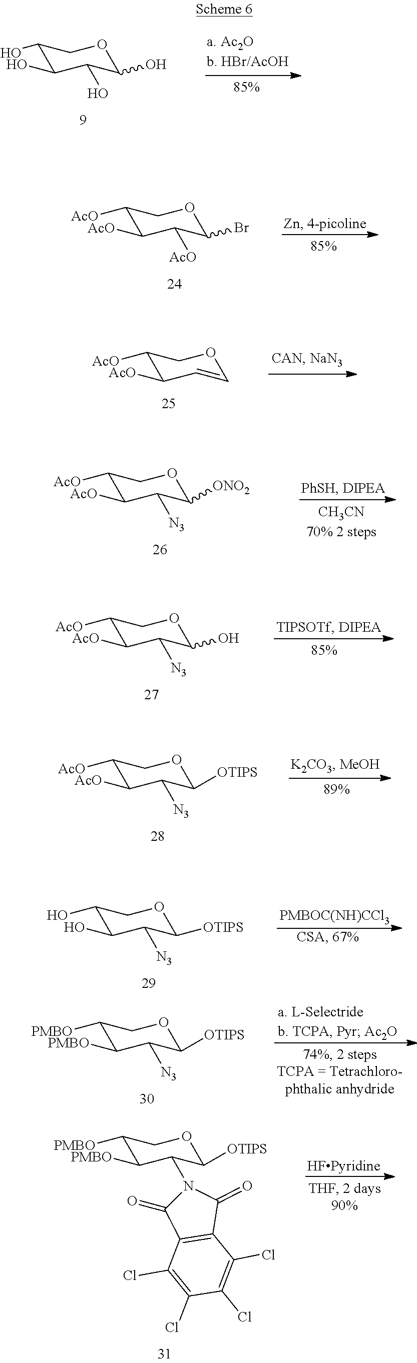

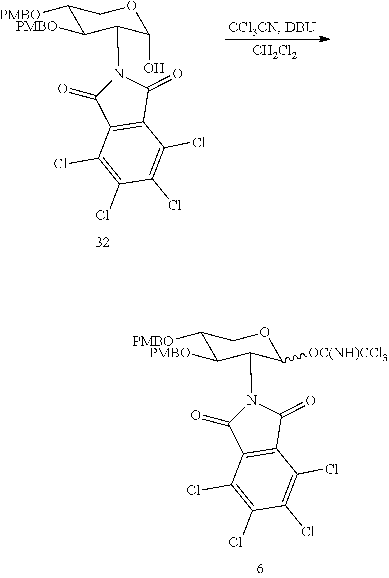

### Synthesis of Representative Compound 4

Sequential couplings of monosaccharide units 8 and 6 to aglycone 7 and completion of the synthesis of compound 4 are shown in Scheme 7. Glycosylation between aglycone 7 and compound 8 in the presence of a catalytic amount of TMSOTf provided compound 33. L-selectride mediated reduction of azide to amine followed by acylation gave compound 34. Chemoselective deprotection of the allyl group by Pd(PPh₃)₄ afforded compound 5. Glycosylation between compound 5 and compound 6 gave compound 35. Removal of the tetrachlorophthalimide protecting group followed by amide formation provided compound 36, which underwent global deprotection by CF₃COOH to give compound 4.

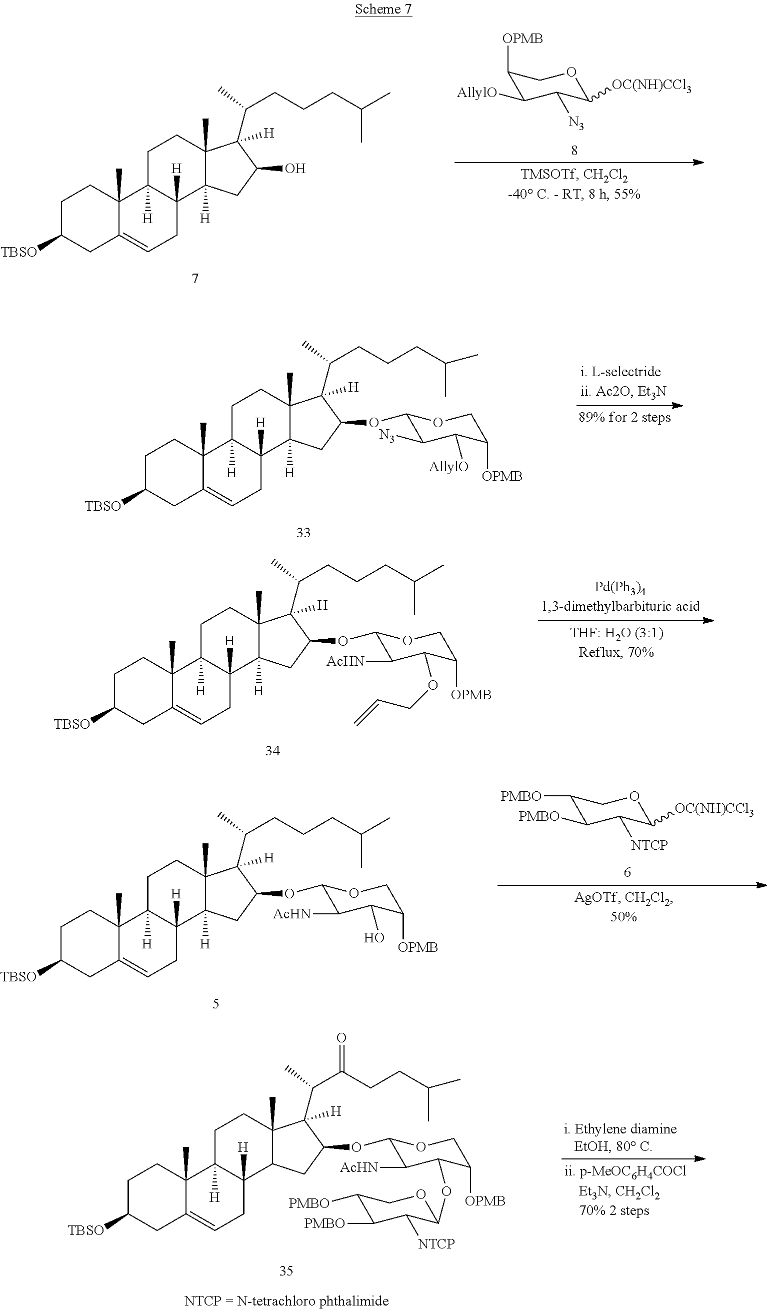

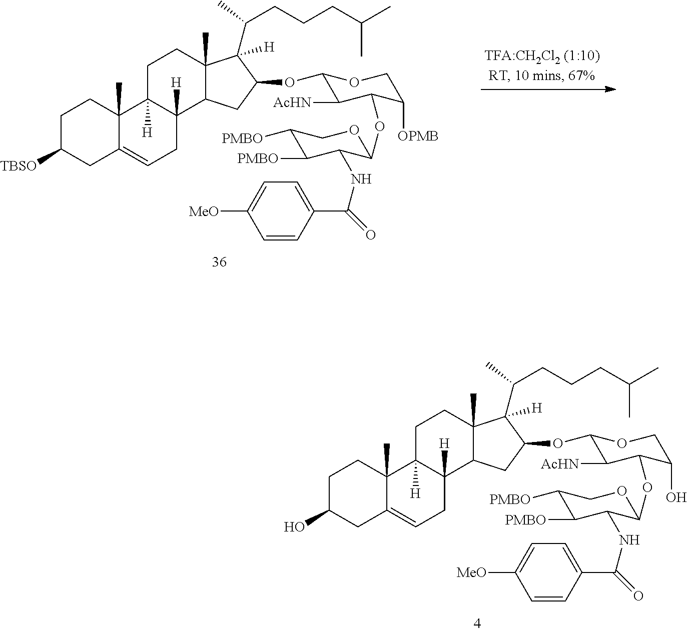

The following definitions are used, unless otherwise described: halo is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

The term saccharide includes monosaccharides, disaccharides, trisaccharides and polysaccharides. The term includes glucose, sucrose, fructose and ribose, as well as deoxy sugars such as deoxyribose and other unnatural sugars. Saccharide derivatives can conveniently be prepared as described in International Patent Applications Publication Numbers WO 96/34005 and 97/03995. A saccharide can conveniently be linked to the remainder of a compound of formula I through an ether (glycosyl) bond.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

A specific value for R¹ is H.

A specific value for R² is H.

A specific value for R³ is H.

A specific value for B is H or OH.

A specific value for B is H.

A specific value for X is acetylamino.

A specific value for Y is 4-methoxybenzoylamino.

A specific value for Z is 4-methyl-1-pentyl.

Processes and intermediates that are useful for preparing compounds of formula (I) are provided as further embodiments of the invention.

A compound of formula (I) can be prepared by adding a solution of CH₂Cl₂:TFA (10:1) (2 ml) to compound 36 (14 mg, 0.010 mmol) at room temperature and stirred for 10 mins. The reaction was quenched with a solution of saturated NaHCO₃ (2 mL), diluted with CH₂Cl₂ (10 mL) and dried over Na₂SO₄. The solution was concentrated under vacuum and purified by column chromatography on silica gel using CH₂Cl₂:MeOH (10:1) as the eluent to obtain the crude product 4 (6 mg) in a 67% yield. The crude product was subject to preparative TLC purification followed HPLC purification. The HPLC purification was performed using a Beckman System Gold instrument with a model 166P variable-wavelength UV detector connected to a 128 solvent module, equipped with a semi-preparative Apollo C18 column (Grace, 1.5×25 cm, 5 μm) under UV detection at 254 nm. 50-100% H₂O/MeOH solvent gradient was used to obtain the pure product 4 with a retention time of 32 mins.

In cases where compounds are sufficiently basic or acidic, a salt of a compound of formula (I) can be useful as an intermediate for isolating or purifying a compound of formula (I). Additionally, administration of a compound of formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to act as anticancer agent may be determined using pharmacological models which are well known to the art, or using Test A described below.

### Test A.

The antiproliferative effect can be determined by performing 3-(4,5-dimethylthiazol-2-yl)-2,5-diphenyltetrazolim (MTT) assay. Briefly, cancer cells at varying concentrations (such as 2,000 cells/well or 5,000 cells/well) will be seeded onto a 96-well plate in triplicates. Following an overnight incubation, the cells will be treated with log-scale serially increasing concentrations of testing compound for 72 hours at 37° C. At the end of the 72 hours period, cells will be treated with the MTT reagent (Sigma-Aldrich, MO) and the antiproliferative effect of testing compound will be measured as previously described (Zhou Y, Achanta G, Pelicano H, Gandhi V, Plunkett W, and Huang P (2002). Molecular Pharmacology 61:222-229). The IC₅₀ values correspond to a concentration of testing compound that inhibits cell viability by 50%.

The invention will now be illustrated by the following non-limiting Examples.

### EXAMPLES

### General Methods

Unless stated otherwise, reactions were performed in flame-dried glassware under a positive pressure of argon using freshly distilled solvent. Tetrahydrofuran (THF) and diethyl ether were distilled from sodium/benzophenone before use. Dichloromethane and toluene were distilled from CaH₂. Anhydrous methanol (99.8%) was purchased from Aldrich. Thin-layer chromatography (TLC) was performed using Dynamic Adsorbents silica gel w/h F₂₅₄ 250 μm glass plates. Visualization of the developed chromatography was performed by UV absorbance (254 nm) and visualizing solutions. The commonly employed TLC visualizing stains were: 12-molybdophosphoric acid solution and Hanessian's stain. Column chromatography was performed using Dynamic absorbents silica gel (32-63 μm).

All ¹H-NMR and ¹³C-NMR spectra were recorded with a Bruker Avance300 (300 MHz) and Bruker Avance600 (600 MHz). In reported ¹H NMR spectrum, data are presented as follows: chemical shift in ppm on the δ scale relative to δH 7.26 for the residual protons in CDCl₃, δH 7.16 for the residual protons in C₆D₆ and δH 3.31 for the residual protons in CD₃OD, integration, multiplicity (s=singlet, d=doublet, t=triplet, q=quartet, m=multiplet, br=broad), and coupling constant (J/Hz). Coupling constants were taken directly from the spectra and are uncorrected. In reported ¹³C NMR spectrum, all chemical shift values are reported in ppm on the δ scale, with an internal reference of δC 77.16 for CDCl₃, δC 128.06 for C₆D₆ and δC 49.00 for CD₃OD. Mass spectral determinations were carried out by using electrospray ionization as ionization source (ESI). Optical rotations were measured on Jasco P-1020 polarimeters.

### Example 1

a. Synthesis of Compound 12

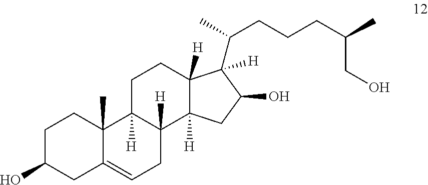

Zinc dust (22.5 g, 0.344 mol) and diosgenin (1.10 g, 2.75 mmol) were mixed in ethanol (250 mL) and the solution was heated to reflux. 19% hydrochloric acid (200 mL) was added to the reaction mixture over 1 hour and the refluxing was continued for further 30 minutes. The unreacted zinc dust was removed by filtration, water (1 L) and diethyl ether (1 L) was added to this solution until the layers separated. The two layers were separated and the aqueous layer was extracted with diethyl ether (3×300 mL). The combined organic layers were washed with a solution of saturated NaHCO₃ till the bubbling stops and dried over magnesium sulfate. Removal of the solvent and recrystallization of the crude product with ethanol/water (1:1) solvent system provided the pure product 12 (810 mg) with 73% yield. ¹H NMR (300 MHz, CD₃OD) δ5.29 (d, J=4.4 Hz, 1H), 4.25 (dd, J=12.3, 7.7 Hz,1H), 3.32-3.22 (m, 2H), 2.19-0.93 (m, 36H), 0.87-0.83 (m, 6H); ¹³C NMR (75 MHz, CD₃OD) δ142.27, 122.33, 72.64, 72.36, 68.57, 62.93, 55.79, 51.66, 43.36, 43.00, 41.33, 38.50, 38.00, 37.69, 37.34, 36.89, 34.73, 32.99, 32.83, 32.27, 31.25, 25.00, 21.90, 19.94, 18.88, 17.13, 13.52.

b. Synthesis of Compound 13

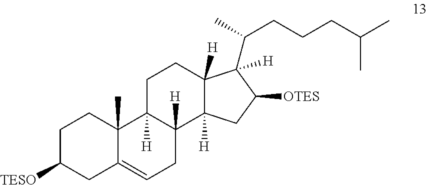

To a solution of triol 12 (2.00 g, 4.77 mmol) and Et₃SiH (7.63 mL, 47.7 mmol) in dry CH₂Cl₂ (16 mL) was added B(C₆F₅)₃ (122 mg, 0.238 mmol) at 0° C. The reaction was stirred at 0° C. for 2 hours and gradually warmed to room temperature and stirred for 6 days. Et₃N (3 mL) was added to quench the reaction and the solvent was removed under pressure. The crude residue was purified by column chromatography on silica gel using hexane-EtOAc (50:1) as the eluent to give the crude product 13 (2.6 g, 86% crude yield). This crude mixture of the desired product was directly used for the next reaction.

c. Synthesis of Compound 14

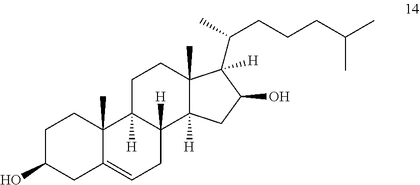

To a solution of crude compound 13 (2.6 g, 4.11 mmol) in CH₂Cl₂ and MeOH (60 mL, 1:1) was added p-toluene sulfonic acid monohydrate (35.4 mg, 0.205 mmol) at rt. The reaction was stirred overnight, cooled to 0° C. and quenched with saturated NaHCO₃ (100 mL) The reaction was diluted with CH₂Cl₂ (100 mL) and the organic phase was separated. The aqueous phase was extracted with CH₂Cl₂ (50 mL×3). The combined organic phase was washed with brine and dried over Na₂SO₄. The filtrate was concentrated under vacuum and purified by column chromatography on silica gel using hexane-EtOAc (5:1-3:1) as the eluent to obtain the pure diol 14 (1.215 g) in a 63% yield for two steps. ¹H NMR (300 MHz, CDCl₃) δ5.35 (d, J=4.7 Hz, 1H), 4.36 (m, 1H), 3.61-3.42 (m, 1H), 2.27-2.20 (m, 2H), 2.02-1.97 (m, 2H), 1.85-1.81 (m, 3H), 1.59-1.40 (m, 9H), 1.28-0.83 (m, 25H); ¹³C NMR (75 MHz, CDCl₃) δ140.56, 121.19, 72.22, 71.46, 61.06, 54.17, 49.76, 41.95, 41.87, 39.52, 39.17, 36.87, 36.20, 35.97, 31.50, 31.30, 31.16, 29.49, 27.78, 26.14, 22.51, 22.28, 20.39, 19.10, 17.92, 13.86, 12.73.

d. Synthesis of Compound 7

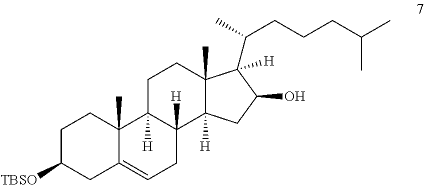

To a solution of diol 14 (80 mg, 0.19 mmol) in CH₂Cl₂(2 mL) was added imidazole (20 mg, 0.29 mmol) and TBSC1 (34 mg, 0.228 mmol) sequentially. The solution was stirred at room temperature overnight and quenched with a solution of saturated NaHCO₃ (5 mL). The organic layer was separated and the aqueous layer was extracted with CH₂Cl₂ (50 mL×3). The combined organic phase was washed with brine and dried over Na₂SO₄. The filtrate was concentrated under vacuum and purified by column chromatography on silica gel using hexane-EtOAc (40:1-20:1) as the eluent to obtain the product 7 (91 mg) in an 89% yield. ¹H NMR (300 MHz, CDCl₃) δ5.31 (d, J=4.8 Hz, 1H), 4.35 (m, 1H), 3.56-3.38 (m, 1H), 2.33-2.11 (m, 3H), 2.09-1.91 (m, 2H), 1.78 (m, 2H), 1.61-1.31 (m, 9H), 1.29-0.79 (m, 34H), 0.06 (s, 6H); ¹³C NMR (75 MHz, CDCl₃) δ141.78, 121.07, 72.73, 72.64, 61.50, 54.65, 50.28, 42.91, 42.31, 39.99, 39.61, 37.44, 36.71, 36.59, 36.40, 36.21, 32.17, 31.97, 31.60, 29.92, 28.21, 26.08, 24.33, 22.94, 22.71, 20.80, 19.56, 18.41, 18.36, 13.15, −4.46.

e. Synthesis of L-Arabinose Tetraacetate

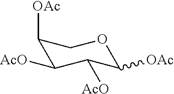 [SMILES: CC(=O)OC1COC(OC(C)=O)C(OC(C)=O)C1OC(C)=O]

To a stirred solution of L-arabinose (5.00 g, 33.3 mmol), Ac₂O (14.14 mL, 149 9 mmol) and Et₃N (21.99 mL, 156.5 mmol) in CH₂Cl₂ (60 mL) at 0° C. was added DMAP (101 mg, 0.83 mmol). The resulting solution was stirred at rt for 12 h and washed with ice water (200 mL×3). The reaction was quenched with a solution of saturated NaHCO₃ and the aqueous phase was extracted with CH₂Cl₂ (100 mL×3). The combined organic phase was washed with brine and dried over Na₂SO₄. The filtrate was concentrated under vacuum affording a sticky residue L-arabinose tetraacetate (10.48 g, 99%), which was used directly in next step without further purification.

f. Synthesis of Compound 15

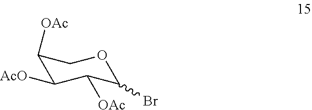 [SMILES: CC(=O)OC1COC(Br)C(OC(C)=O)C1OC(C)=O]

To a solution of L-arabinose tetraacetate (491 mg, 1.57 mmol) in CH₂Cl₂ (10 mL) HBr (33% in AcOH, 0.8 mL, 4.4 mmol) was added slowly at 0° C. The reaction mixture was stirred at 0° C. for 4 hours and diluted with water (5 mL). The organic layer was poured into cold saturated aqueous NaHCO₃ (15 mL) under vigorous stirring. The organic phase was separated, and the aqueous phase was extracted with CH₂Cl₂ (10 mL×3). The combined organic layer was washed with brine and dried over Na₂SO₄. The filtrate was concentrated under vacuum affording a brown oil which was subjected to a quick flash column chromatography using hexane-EtOAc (10:1-5:1) as the eluent to afford bromide 15 as a colorless oil (470 mg, 88%).

g. Synthesis of Compound 16

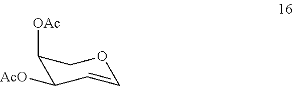 [SMILES: CC(=O)OC1C=COCC1OC(C)=O]

4-picoline (30 μl, 0.29) was added to a suspension of Zn dust (114 mg, 1.74 mmol) in EtOAc (2 mL). The mixture was refluxed with vigorous stirring and a solution of bromide 15 (100 mg, 0.29 mmol) in EtOAc (2 mL) was added over 5 mins. After 45 minutes, the mixture was cooled down and filtered over a pad of celite. The filtrate was washed with 10% aqueous HCl and saturated NaHCO₃, dried over Na₂SO₄ and concentrated under vacuum. The residue was purified by flash column chromatography using hexane-EtOAc (10:1-5:1) as the eluent to afford alkene 16 as a white solid (50 mg, 85%).

h. Synthesis of Compound 17

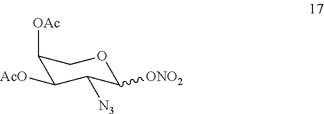

To a solution of alkene 16 (213 mg, 1.06 mmol) in dry acetonitrile (5 mL) at −20° C., NaN₃ was added, followed by the addition of CAN (2.4 g, 4.24 mmol) in two portions. After 2 hours, the reaction mixture was poured into ice water (10 mL). The mixture was extracted with Et₂O (20 mL×3) and the organic phase was washed with water and brine. The crude mixture 17 was used directly for the next step without purification.

i. Synthesis of Compound 18

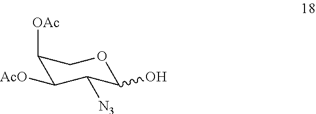 [SMILES: CC(=O)OC1COC(O)C(N=[N+]=[N-])C1OC(C)=O]

To a solution of compound 17 in acetonitrile (5 mL) at 0° C. was added DIPEA (0.28 mL, 1.59 mmol) and PhSH (0.13 mL, 1.27 mmol) sequentially. The resulting mixture was stirred at 0° C. for 2 hours. The reaction mixture was concentrated under vacuum to give a brown sticky oil which was purified by column chromatography using hexane-EtOAc (5:1-3:1) as the eluent to afford lactol 18 as a colorless oil (178 mg, 65% for two steps).

### Synthesis of Compound 19

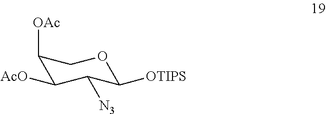

To a stirred solution of lactol 18 (1 g, 3.85 mmol) in CH₂Cl₂ (15 mL) at 0° C. was added 2,6-luitidine (670 μL, 5.78 mmol) and TIPSOTf (1.244 mL, 4.629 mmol) sequentially. The resulting solution was stirred at room temperature for 6 h, diluted with hexanes and filtered over a pad of celite. The filtrate was concentrated under vacuum giving a residue. The crude product 19 from this reaction was directly used to the next reaction without extensive purification.

k. Synthesis of Compound 20

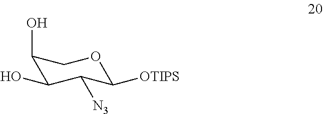

To a stirred solution of crude compound 19 (1 g, 2.408 mmol) in methanol (10 mL) at 0° C. was added K₂CO₃ (83 mg, 0.602 mmol). The resulting solution was stirred at rt for overnight and quenched with a solution of saturated NH₄Cl (30 mL). The methanol was removed under vacuum and the crude solid was partitioned in EtOAc (150 mL) and water (150 mL). The aqueous phase was extracted with EtOAc (150 mL×3), washed with water and brine and concentrated under vacuum. The residue was purified by flash column chromatography using hexane-EtOAc (10:1-5:1) as the eluent to afford diol 20 (1.01 g, 79% for 2 steps) as a colorless oil. [α]_{D}¹⁹=+56.33 (c=1.00 in CHCl₃); ¹H NMR (300 MHz, CDCl₃) δ4.52 (d, J=6.3 Hz, 1H), 3.98 (dd, J=12.8, 2.7 Hz, 1H), 3.88 (br, 1H), 3.55-3.40 (m, 4H), 3.22 (d, J=5.1 Hz, 1H), 1.07 (m, 21H); ¹³C NMR (75 MHz, CDCl₃) δ97.16, 67.46, 66.60, 65.43, 17.64, 17.62, 12.08; HR-MS (ESI): calcd for C₁₄H₂₉N₃NaO₄Si⁺[M+Na⁺]: 354.1825, found: 354.1826.

l. Synthesis of Compound 21

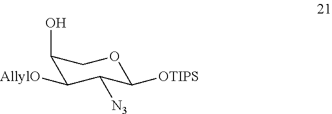

The suspension of diol 20 (220 mg, 0.663 mmol) and Bu₂SnO (231 mg, 0.929 mmol) in anhydrous toluene (30 mL) was refluxed for 24 hour with azeotropic removal of water. After 24 hours, the solution was evaporated to approximate 5 mL and cooled to 60° C. Tetrabutylammonium iodide (367 mg, 0.995 mmol) and allyl bromide (0.572 mL, 6.63 mmol) were added to the reaction mixture. The resulting mixture was stirred at 60° C. with a reflux condenser attached for 5 hours. On the consumption of starting material, the reaction mixture was diluted with CH₂Cl₂ (50 mL) and filtered over a pad of celite. The filtrate was concentrated under vacuum giving a residue which was subjected to flash column chromatography using hexane-EtOAc (10:1-5:1) as the eluent to afford alcohol 21 (217 mg, 88%) as a colorless oil. [α]_{D}¹⁹ =+18.33 (c=1.00 in CHCl₃); ¹H NMR (300 MHz, CDCl₃) δ5.94 (m, 1H), 5.28 (ddd, J=13.8, 11.7, 1.5 Hz, 2H), 4.43 (d, J=7.5 Hz, 1H), 4.21-4.08 (m, 2H), 4.04 (dd, J=13.0, 2.0 Hz, 1H), 3.90 (br, 1H), 3.58 (dd, J=5.3, 3.5 Hz, 1H), 3.49 (dd, J=10.0, 7.5 Hz, 1H), 3.39 (d, J=13.0, 5.3 Hz, 1H), 3.23 (dd, J=10.0, 3.5 Hz, 1H), 1.08 (m, 21H); ¹³C NMR (75 MHz, CDCl₃) δ134.17, 118.28, 97.53, 78.63, 71.30, 65.72, 65.51, 17.85, 17.83, 12.33; HR-MS (ESI): calcd for C₁₇H₃₃N₃NaO₄Si⁺[M+Na]: 394.2138, found: 394.2143.

m. Synthesis of Compound 22

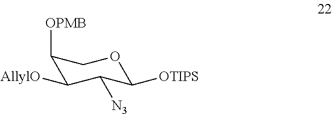

To dry alcohol 21 (220 mg, 0.592 mmol) at 0° C., was added a 1.0 M solution of PMB trichloroimidate (0.88 μL, 0.88 mmol) dropwise, followed by addition of CSA (20 mg, 0.088 mmol). The reaction mixture was stirred for overnight at room temperature. The reaction mixture was diluted with hexanes (15 mL) and filtered over a pad of celite. The filtrate was concentrated under vacuum to give crude product, which was subjected to flash chromatography using hexane-EtOAc (20:1-10:1) as the eluent to afford the product 22 (234 mg, 80%) as a colorless oil. [α]_{D}¹⁹=+79.25 (c=1.00 in CHCl₃); ¹H NMR (300 MHz, CDCl₃) δ7.31 (d, J=8.7 Hz, 2H), 6.88 (d, J=8.7 Hz, 2H), 5.91 (m, 1H), 5.28 (ddd, J=13.8, 11.7, 1.5 Hz, 2H), 4.66 (dd, J=25.7, 12.2 Hz, 2H), 4.42 (d, J=7.5 Hz, 1H), 4.02 (m, 3H), 3.80 (s, 3H), 3.71-3.59 (m, 2H), 3.23-3.13 (m, 2H), 1.08 (m, 21H); ¹³C NMR (75 MHz, CDCl₃) δ159.35, 134.60, 130.24, 129.59, 117.54, 113.86, 97.73, 78.87, 70.93, 70.87, 70.75, 65.92, 63.37, 55.40, 17.89, 17.88, 12.40; HR-MS (ESI): calcd for C₂₅H₄₁N₃NaO₅Si⁺[M+Na⁺]: 514.2713, found: 514.2720.

n. Synthesis of Compound 23

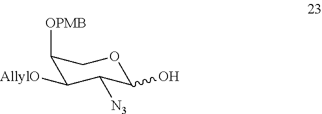

To a solution of compound 22 (234 mg, 0.475 mmol) and pyridine (2 mL) in THF (5 mL) at 0° C. in a plastic container, HF/pyridine (70% solution) (1 mL) was added dropwise. The reaction was stirred at 0° C. for 30 minutes and warmed up to rt and stirred for 48 hours. Saturated aqueous NaHCO₃ (5 mL) was added to quench the reaction, and EtOAc (20 mL) was added to dilute the mixture. The organic phase was separated and the aqueous phase was extracted with EtOAc (10 mL×3). The combined organic layers were washed with brine, dried over Na₂SO₄, and concentrated under vacuum. The residue was purified by flash column chromatography using hexane-EtOAc (5:1-3:1) as the eluent to give the lactol 23 (147 mg, 92%). [α]_{D}¹⁹=+9.5 (c=1.00 in CHCl₃); ¹H NMR (300 MHz, CDCl₃) δ7.30 (d, J=7.1 Hz, 2H), 6.88 (d, J=7.1 Hz, 2H), 6.01-5.84 (m, 1H), 5.39-5.11 (m, 311), 4.66-4.47 (m, 3H), 4.18-3.95 (m, 3H), 3.95-3.83 (m, 2H), 3.80 (s, 3H), 3.41-3.31 (m, 1H); ¹³C NMR (75 MHz, CDCl₃) δ159.39, 159.32, 134.21, 134.03, 129.66, 129.57, 117.93, 117.69, 113.87, 113.84, 96.01, 92.50, 78.58, 77.06, 75.26, 71.37, 71.26, 70.59, 70.35, 63.66, 62.73, 60.47, 60.10, 55.31; HR-MS (ESI): calcd for C₁₆H₂₁N₃NaO₅⁺[M+Na⁺]: 358.1379, found: 358.1384.

o. Synthesis of D-Xylose Tetraacetate

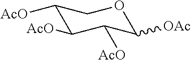 [SMILES: CC(=O)OC1COC(OC(C)=O)C(OC(C)=O)C1OC(C)=O]

To a stirred solution of D-xylose (30 g, 200 mmol), Ac₂O (75.62 mL, 800 mmol) and Et₃N (139.57 mL, 1000 mmol) in CH₂Cl₂ (1 L) was added DMAP (1.2 g, 10 mmol) at 0° C. The resulting solution was stirred at room temperature for 12 hours and washed with ice water (1 L×3). The reaction was quenched with a solution of saturated NaHCO₃ and the aqueous phase was extracted with CH₂Cl₂ (500 mL×3). The combined organic phase was washed with brine and dried over Na₂SO₄. The filtrate was concentrated under vacuum affording a sticky residue D-xylose tetraacetate (60.42 g, 95%), which was used directly in next step without further purification.

p. Synthesis of Compound 24

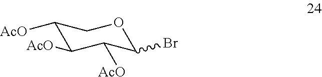 [SMILES: CC(=O)OC1COC(Br)C(OC(C)=O)C1OC(C)=O]

To a solution of D-xylose tetraacetate (5.00 g, 16.0 mmol) in CH₂Cl₂(50 mL) HBr (33% in AcOH, 9.87 mL, 44.7 mmol) was added slowly at 0° C. The reaction mixture was stirred at 0° C. for 4 hours and diluted with water (40 mL). The reaction mixture was poured into cold saturated aqueous NaHCO₃ (150 mL). The organic layer was separated and aqueous phase was extracted with CH₂Cl₂ (100 mL×3). The combined organic layer was washed with brine and dried Na₂SO₄. The filtrate was concentrated under vacuum giving a residue which was subjected to flash column chromatography using hexane-EtOAc (10:1-5:1) as the eluent to afford bromide 24 (4.62 g, 85%).

q. Synthesis of Compound 25

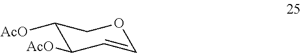 [SMILES: CC(=O)OC1C=COCC1OC(C)=O]

4-picoline (300 μl, 2.99 mmol) was added to a suspension of Zn dust (1.14 g, 17.4 mmol) in EtOAc (5 mL). The mixture was refluxed with vigorous stirring and a solution of bromide 24 (1.0 g, 2.90 mmol) in EtOAc (10 mL) was added over 10 mins. After 45 minutes, the mixture was cooled down and filtered over a pad of celite. The filtrate was washed with saturated aqueous NaHCO₃ and brine, dried over Na₂SO₄ and concentrated under vacuum. The residue was purified by flash column chromatography using hexane-EtOAc (10:1-5:1) as the eluent to afford alkene 25 (482 mg, 85%).

r. Synthesis of Compound 26

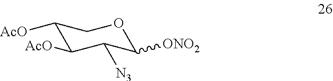

To a solution of alkene 25 (4.6 g, 23.2 mmol) in dry acetonitrile (150 mL) at −20° C., NaN₃ (4.5 g, 69.4 mmol) was added, followed by addition of CAN (50.76 g, 92.5 mmol) in two portions. After 2 hours, the reaction was poured into ice water (100 mL). The mixture was extracted with Et₂O (100 mL×3) and the organic phase was washed with water and brine. The crude mixture 26 was used directly without further purification in the next step.

s. Synthesis of Compound 27

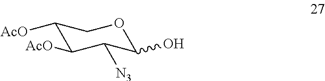 [SMILES: CC(=O)OC1COC(O)C(N=[N+]=[N-])C1OC(C)=O]

To a solution of compound 26 in acetonitrile (150 mL) at 0° C. was added DIPEA (4.8 mL, 34.8 mmol) and PhSH (2.0 mL, 25.5 mmol) sequentially. The reaction was stirred for 2 hours at 0° C. and concentrated under vacuum. The residue was purified by flash column chromatography Hexane-EtOAc (5:1-3:1) as the eluent to afford lactol 27 (3.89 g, 70% from two steps) as a clear oil. ¹H NMR (300 MHz, CDCl₃): δ5.51 (t, J=9.6 Hz, 1H), 5.32 (d, J=3.2 Hz, 1H), 5.05-4.91 (m, 311), 4.66 (d, J=8.0 Hz, 1H), 4.07 (q, J=5.6 Hz, 1H), 3.92-3.78 (m, 4H), 3.43 (q, J=8.0, 9.6 Hz, 1H), 3.33 (m, 111); ¹³C NMR (75 MHz, CDCl₃): δ171.3, 171.23, 171.22, 171.19, 97.8, 93.2, 94.1, 91.0, 70.3, 70.1, 65.8, 63.9, 62.6, 59.8, 21.83, 21.81, 21.77, 21.71.

t. Synthesis of Compound 28

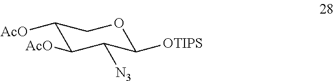

To a solution of lactol 27 (486 mg, 1.88 mmol) and DIPEA (654 μL, 3.76 mmol) in CH₂Cl₂ (10 mL), TIPSOTf (760 μL, 2.82 mmol) was added dropwise at 0° C. The reaction was warmed to room temperature and stirred for 2 hours and quenched with water. The aqueous phase was separated and extracted with CH₂Cl₂ (20 mL×3). The combined organic phase was washed with brine, dried over Na₂SO₄ and concentrated under vacuum. The residue was purified by flash column chromatography hexane-EtOAc (10:1-5:1) as the eluent to afford compound 28 (662 mg, 85%) as pale yellow oil. ¹H NMR (300 MHz, CDCl₃): δ4.92-4.76 (m, 2H), 4.53 (d, J=7.2 Hz, 1H), 3.96 (dd, J=11.5, 5.1 Hz, 1H), 3.26 (dd, J=9.6, 7.2 Hz, 1H), 3.15 (dd, J=9.6, 11.5 Hz, 1H), 2.00 (s, 3H), 1.91 (s, 3H), 1.03-0.92 (m, 21H); ¹³C NMR (75 MHz, CDCl₃) δ169.63, 169.58, 97.16, 71.47, 68.90, 66.13, 62.22, 20.45, 20.35, 17.39, 17.35, 11.87; HR-MS (ESI): calcd for C₁₈H₃₃N₃O₆Na Si⁺[M+Na⁺]: 438.2031, found: 438.2034.

u. Synthesis of Compound 29

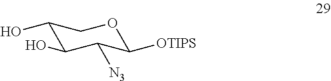

To a solution of compound 28 (3.1 g, 7.45 mmol) in methanol (50 mL) at room temperature was added anhydrous K₂CO₃ (258 mg, 1.86 mmol). The reaction was stirred at room temperature overnight, and quenched with a solution of saturated NH₄Cl (30 mL) and diluted with EtOAc (10 mL) The methanol was removed under vacuum and the crude solid was partitioned in EtOAc (150 mL) and water (150 mL). The aqueous phase was extracted with EtOAc (150 mL×3), washed with water and brine and concentrated under vacuum. The filtrate was concentrated under vacuum to afford the reaction crude, which was purified by flash chromatography using hexane-EtOAc (5:1-2:1) as the eluent to afford diol 29 (2.2 g, 89%) as a white solid. [α]_{D}¹⁸=+4.66 (c=1.00 in CHCl₃);¹H NMR (300 MHz, CDCl₃): δ4.55 (d, J=7.2 Hz, 1H), 4.45 (m, 1H), 4.29 (m, 1H), 3.94 (dd, J=11.7, 5.4 Hz, 1H), 3.75-3.60 (m, 1H), 3.28 (t, J=9.6 Hz, 1H), 3.22-3.10 (m, 2H), 1.20-1.02 (m, 21H); ¹³C NMR (75 MHz, CDCl₃): δ97.7, 74.8, 69.8, 68.7, 65.3, 17.6, 12.1; HR-MS (ESI): calcd for C₁₄H₂₉N₃O₄SiNa⁺[M+Na⁺]: 354.1825, found: 353.1824.

v. Synthesis of Compound 30

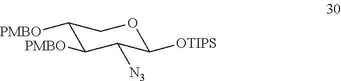

To dry diol 29 (549 mg, 1.65 mmol) at 0° C., was added a 1.0 M solution of PMB trichloroimidate (4.96 mL, 4.96 mmol) dropwise, followed by addition of CSA (76 mg, 0.33 mmol). The reaction mixture was stirred for overnight at room temperature. The reaction mixture was diluted with hexanes (15 mL) and filtered over a pad of celite. The filtrate was concentrated under vacuum to give reaction crude, which was subjected to flash chromatography using hexane-EtOAc (30:1-20:1) as the eluent to afford the product 30 (634 mg, 67%) as a colorless oil. [α]_{D}²⁰=−37.9 (c=1.00 in CHCl₃); ¹H NMR (300 MHz, CDCl₃): δ7.25 (d, J=8.7 Hz, 2H), 7.17 (d, J=8.7, 2H), 6.82 (d, J =8.7 Hz, 2H), 6.80 (d, J=8.7 Hz, 2H), 4.76-4.65 (m, 2H), 4.61-4.44 (m, 2H), 4.41 (d, J=7.2 Hz, 1H), 3.85 (dd, J=11.7, 5.4 Hz, 1H), 3.73 (s, 3H), 3.72 (s, 3H), 3.53 (ddd, J=10.2, 8.4, 5.4 Hz, 1H), 3.30-3.12 (m, 2H), 3.05 (t, J=11.4 Hz, 1H), 1.12-0.94 (m, 21H); ¹³C NMR (75 MHz, CDCl₃): δ159.5, 159.4, 130.5, 130.2, 129.8, 129.5, 114.0, 113.9, 97.5, 81.9, 77.6, 75.1, 73.0, 69.0, 64.0, 61.5, 55.3, 17.8, 17.8, 12.3; HR-MS (ESI): calcd for C₃₀R₄₅N₃NaO₆Si⁺[M+Na⁺]: 594.2975, found: 594.2974.

w. Synthesis of Compound 31

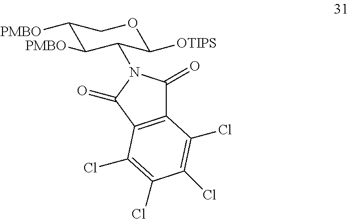

To a solution of azide 30 (184 mg, 0.32 mmol) in dry THF (2 mL) under Argon atmosphere, L-Selectride (1.28 mL, 1.28 mmol, 1.0 M in THF) was added dropwise at −78° C. The resulting mixture was warmed up to rt and stirred for 5 hours. On consumption of azide, the reaction was quenched by adding saturated aqueous K⁺Na⁺Tartarate (5 mL) and diluted with EtOAc (5 mL). The organic layer was separated and the aqueous layer was extracted with EtOAc (10 mL×3). The combined organic layer was washed with brine and dried over Na₂SO₄. After filtration, the solvent was removed and the crude product was used directly in next step without purification.

To a solution of crude amine in pyridine (2 mL) under argon atmosphere at 0° C., TCPA (183 mg, 0.64 mmol) was added. After the resulting solution was stirred at room temperature for 1 hour, Ac₂O (302 μL, 3.2 mmol) was added. The reaction mixture was then heated at 80° C. overnight. Solvent was removed under vacuum to give a brown residue, which was subjected to flash chromatography column using hexane-EtOAc (20:1-10:1) as the eluent to compound 31 (194 mg, 74% for 2 steps). [α]_{D}²⁰=+43.1 (c =1.00 in CHCl₃);¹H NMR (300 MHz, CDCl₃): δ7.30 (d, J=8.5 Hz, 2H), 6.97 (d, J=8.5 Hz, 2H), 6.90 (d, J=8.5 Hz, 2H), 6.40 (d, J=8.5 Hz, 2H), 5.33 (d, J=7.8 Hz, 1H), 4.79 (d, J=12.6 Hz, 1H), 4.71 (d, J=11.4 Hz, 1H), 4.63 (d, J=11.4 Hz, 1H), 4.34 (d, J=12.6 Hz, 1H), 4.10 (dd, J=10.8, 8.7 Hz, 1H), 3.97 (dd, J=11.7, 5.4 Hz, 1H), 3.90 (d, J=10.8, 7.8 Hz,1H), 3.81 (s, 3H), 3.70 (m, 1H), 3.58 (s, 3H), 3.29 (t, J=11.1 Hz, 1H), 1.0-0.8 (m, 21H); ¹³C NMR (75 MHz, CDCl₃) δ159.50, 158.64, 131.12, 130.34, 129.94, 129.57, 114.04, 113.88, 113.13, 93.64, 79.36, 78.68, 74.57, 73.20, 71.56, 64.00, 58.83, 55.41, 17.67, 12.07; HR-MS (ESI): calcd for C₃₈H₄₅C₁₄NNaO₈Si⁺[M+Na⁺]: 834.1566, found: 834.1562.

x. Synthesis of Compound 32

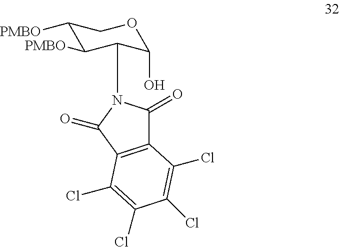

To a solution of compound 31 (73 mg, 0.089 mmol) and pyridine 250 (μL) in THF (1.5 mL) at 0° C. in a plastic container, HF/pyridine (70% solution) (120 μL) was added dropwise. The reaction was stirred at 0° C. for 30 minutes and warmed up to room temperature and stirred for 48 hours. Saturated aqueous NaHCO₃ (5 mL) was added to quench the reaction, and EtOAc (20 mL) was added to dilute the mixture. The organic phase was separated and the aqueous phase was extracted with EtOAc (10 mL×3). The combined organic layers were washed with brine, dried over Na₂SO₄, and concentrated under vacuum. The residue was purified by flash column chromatography using Hexane-EtOAc (5:1-2:1) as the eluent to give the lactol 32 (53 mg, 90%) as a pale yellow oil. [α]_{D}¹⁹=+69.5 (c=1.00 in CHCl₃); ¹H NMR (300 MHz, CDCl₃): 7.21 (d, J=8.7 Hz, 2H), 6.86 (d, J=8.4 Hz, 2H), 6.80 (d, J=8.7 Hz, 2H), 6.29 (d, J=8.4 Hz, 2H), 5.17 (d, J=8.1 Hz, 1H), 4.70 (d, J=12.9 Hz, 1H), 4.58 (dd, J=25.0, 11.3 Hz, 2H), 4.23 (d, J=12.6 Hz, 1H), 4.02 (dd, J=8.4, 10.5 Hz, 1H), 3.88 (dd, J=5.4, 11.4 Hz, 1H), 3.77 (dd, J=8.4, 10.5 Hz, 1H), 3.71 (s, 3H), 3.64-3.57 (m, 1H), 3.47 (s, 3H), 3.29-3.22 (m, 2H); ¹³C HNMR (75 MHz, CDCl₃): δ159.7, 158.8, 131.1, 130.3, 130.0, 129.7, 114.2, 113.3, 93.2, 79.2, 78.8, 74.8, 73.4, 64.5, 58.2, 55.5, 55.2; HR-MS (ESI): calcd for C₂₉H₂₅C₁₄NNaO₈⁺[M+Na⁺]: 678.0232, found: 678.0236.

y. Synthesis of Compound 33

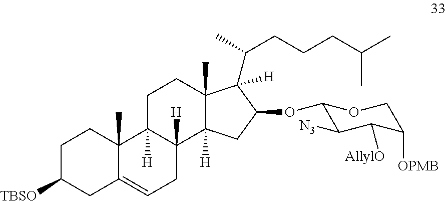

To a solution of compound 23 (200 mg, 0.59 mmol) in dry CH₂Cl₂ (2.5 mL) was added CCl₃CN (70 μL, 0.71 mmol) and DBU (4.3 μL, 0.029 mmol) sequentially and stirred overnight. The aglycone 7 (448 mg, 0.89 mmol) was added to this solution followed by 4 Å molecular sieve and stirred at rt for 2 h. This mixture was cooled to −40° C., and a solution of TMSOTf (21.3 μL, 0.118 mmol) diluted in CH₂Cl₂ (250 μL) was added dropwise over a period of 1 hour. The reaction mixture was warmed to room temperature slowly overnight. The reaction mixture was quenched by Et₃N (2 mL) and filtered over a pad of celite. The filtrate was concentrated under vacuum to give a residue, which was subjected to flash column chromatography using hexane-EtOAc (50:1) as the eluent to afford compound 33 (273 mg, 55%). [α]_{D}¹⁹ =+4.75 (c=1.0 in CHCl₃); ¹H NMR (300 MHz, CDCl₃) δ7.31 (d, J=8.6 Hz, 2H), 6.88 (d, J=8.6 Hz, 2H), 5.91 (ddd, J=22.7, 10.8, 5.6 Hz, 1H), 5.32-5.15 (m, 3H), 4.67 (dd, J=25.7, 12.3 Hz, 2H), 4.11-3.98 (m, 4H), 3.96 (m, 1H), 3.80 (s, 3H), 3.69 (dd, J=10.0, 7.7 Hz, 1H), 3.62 (m, 1H), 3.47 (m, 1H), 3.20 (d, J=13.0 Hz, 2H), 2.35-1.92 (m, 6H), 1.85-1.39 (m, 11H), 1.38-0.94 (m, 33H), 0.05 (s, 6H); ¹³C NMR (75 MHz, CDCl₃) δ159.39, 141.72, 134.53, 130.23, 129.70, 121.18, 117.56, 113.89, 104.60, 82.45, 79.31, 77.36, 72.80, 71.20, 71.00, 70.51, 63.80, 63.70, 61.89, 55.41, 55.17, 50.38, 42.93, 42.37, 40.14, 39.87, 37.45, 36.68, 36.41, 32.31, 32.19, 31.92, 31.52, 30.43, 30.11, 28.30, 26.08, 24.13, 23.09, 22.88, 22.71, 20.98, 19.54, 18.42, 18.17, 14.33, 12.91, −4.46; HR-MS (ESI): calcd for C₄₉H₇₉N₃NaO₆Si⁺[M+Na⁺]: 856.5636, found: 856.5625.

z. Synthesis of Compound 34

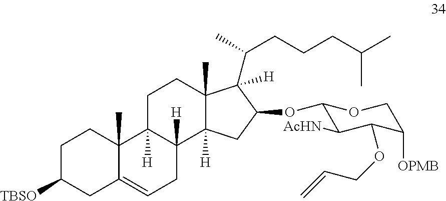

To a well stirred solution of azide 33 (220 mg, 0.263 mmol) in dry THF (3 mL) at −78° C. under Argon, was added L-selectride (1.3 mL, 1.0 M in THF) dropwise. The resulting mixture was warmed up to rt and stirred for 5 h. On consumption of azide, the reaction was quenched with saturated aqueous K⁺Na⁺Tartarate (5 mL) and diluted with EtOAc (5 mL). The organic layer was separated and the aqueous layer was extracted with EtOAc (10 mL×3). The combined organic layer was washed with brine and dried over Na₂SO₄. After filtration, the solvent was removed and the crude amine product was used directly in next step without purification.

To a solution of the above crude amine compound and Et₃N (183 μL, 1.31 mmol) in dry CH₂Cl₂ (5 mL) was added Ac₂O (75 μL, 0.789 mmol) dropwise at 0° C. The resulting mixture was warmed to room temperature and stirred overnight. Saturated aqueous NaHCO₃ (5 mL) was added to quench the reaction, and CH₂Cl₂ (20 mL) was added to dilute the mixture. The organic phase was separated and the aqueous phase was extracted with CH₂Cl₂ (20 mL×3). The combined organic layers were washed with brine, dried over Na₂SO₄, and concentrated under vacuum. The residue was purified by flash column chromatography using hexane-EtOAc (5:1-3:1) as the eluent to afford the amide 34 (200 mg, 89%) as a colorless oil. [α]_{D}²⁰ =−28.9 (c=1.4 in CHCl₃); NMR (300 MHz, CDCl₃) δ7.27 (d, J=8.5 Hz, 2H), 6.86 (d, J=8.5 Hz, 2H), 5.92 (ddd, J=16.2, 10.9, 5.8 Hz, 1H), 5.48 (d, J=7.7 Hz, 1H), 5.35-5.15 (m, 3H), 4.55 (m, 2H), 4.20-3.93 (m, 6H), 3.79 (s, 3H), 3.68-3.33 (m, 3H), 2.29-1.91 (m, 9H), 1.82-1.40 (m, 11H), 1.39-0.88 (m, 33H), 0.05 (s, 6H); ¹³C NMR (75 MHz, CDCl₃) δ169.27, 159.37, 141.70, 134.81, 130.19, 129.62, 121.12, 117.62, 113.86, 101.84, 82.14, 77.36, 73.15, 72.78, 70.55, 70.35, 70.20, 61.37, 60.04, 55.40, 55.05, 51.11, 50.31, 42.92, 42.30, 40.00, 39.78, 37.44, 36.68, 36.36, 35.40, 32.17, 31.91, 31.56, 30.40, 30.01, 28.22, 26.08, 24.02, 23.60, 22.90, 22.74, 20.92, 19.51, 18.42, 18.27, 14.32, 13.03, -4.46; HR-MS (ESI): calcd for C₅₁H₈₃NNaO₇Si⁺[M+Na⁺]: 872.5837, found: 872.5827.

aa. Synthesis of Compound 5

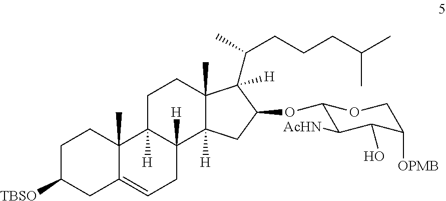

To a stirred solution of compound 34 (80 mg, 0.094 mmmol) in degassed THF:H₂O (5 mL, 3:1 THF:H₂O) was added 1,3-dimethyl barbituric acid (44 mg, 0.28 mmol) and Pd(PPh₃)₄ (21 mg, 0.018 mmol). The solution was refluxed for 5 hours at 90° C. till all the starting material was consumed. On completion of the reaction, the solvent was evaporated under vacuum and the crude residue was subject to flash chromatography using CH₂Cl₂-MeOH (50:1-30:1) as the eluent to obtain compound 5 (54 mg, 71%). [α]_{D}²⁰=−22.0(c=0.83 in CHCl₃); ¹H NMR (300 MHz, CDCl₃) δ7.28 (d, J=8.7 Hz, 2H), 6.87 (d, J=8.7 Hz, 2H), 5.46 (d, J=7.6 Hz, 1H), 5.29 (d, J=4.5 Hz, 1H), 4.59 (dd, J=23.5, 11.7 Hz, 2H), 4.43 (d, J=3.5 Hz, 1H), 4.15-4.08 (m, 1H), 4.06-3.87 (m, 4H), 3.80 (s, 3H), 3.56 (m, 2H), 3.47 (m, 2H), 2.29-1.97 (m, 9H), 1.97-1.42 (m, 14H), 1.40-0.88 (m, 33H), 0.05 (s, 6H);¹³C NMR (75 MHz, CDCl₃) δ169.97, 159.46, 141.80, 130.04, 129.70, 120.95, 113.96, 102.50, 83.10, 72.75, 71.78, 70.81, 68.84, 61.18, 55.43, 54.96, 53.34, 50.25, 42.91, 42.27, 40.05, 39.64, 37.42, 36.68, 35.57, 32.16, 31.87, 31.51, 30.28, 28.20, 26.08, 23.92, 23.56, 22.91, 22.71, 20.87, 19.53, 18.42, 18.24, 14.30, 13.14, −4.46; HR-MS (ESI): calcd for C₄₈H₇₉NNaO₇Si⁺[M+Na⁺]: 832.5524, found: 832.5524.

ab. Synthesis of Compound 35

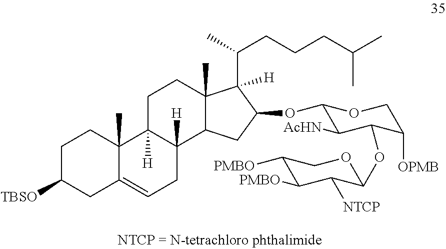

To a solution of compound 32 (100 mg, 0.157 mmol) in dry CH₂Cl₂ (2 mL) was added CCl₃CN (17 μL, 0.167 mmol) and DBU (1.2 μL, 0.008 mmol) sequentially and stirred overnight. Compound 5 (50 mg, 0.062 mmol) was added to this solution followed by 4 Å molecular sieve and stirred at rt for 2 h. This mixture was cooled to 0° C., and AgOTf (10 mg, 0.039 mmol) was added to this mixture. The reaction mixture was warmed to room temperature slowly, protected from light and stirred for 8 h. On completion, the reaction was quenched by Et₃N (2 mL) and filtered over a pad of celite. The filtrate was concentrated under vacuum to give a residue, which was subjected to flash column chromatography using hexane-EtOAc (5:1-3:1) as the eluent to afford compound 35 (44 mg, 50%). [α]_{D}²⁰=+17.2 (c=1.0 in CHCl₃); ¹H NMR (600 MHz, CDCl₃) δ7.32 (d, J=8.0 Hz, 2H), 7.28 (m, 2H), 6.94 (d, J=8.0 Hz, 1H), 6.90 (d, J=7.8 Hz, 1H), 6.85 (d, J=8.2 Hz, 1H), 6.41 (d, J=7.8 Hz, 2H), 5.28 (d, J=3.1, 1H), 5.12 (m, 2H), 4.85-3.99 (m, 10H), 3.83 (s, 9H), 3.74-3.18 (m, 9H), 2.29-1.69 (m, 9H), 1.62-1.14 (m, 13H), 1.17-0.50 (m, 30H), 0.04 (s, 6H). ¹³C NMR (151 MHz, CDCl₃) δ159.51, 159.06, 158.59, 131.00, 130.90, 130.28, 129.83, 129.74, 129.72, 129.67, 129.53, 114.07, 114.01, 113.62, 113.20, 113.14, 101.84, 100.08, 98.77, 98.42, 94.34, 81.43, 79.40, 78.85, 78.80, 78.55, 78.49, 75.59, 74.73, 74.67, 74.60, 73.21, 72.71, 64.91, 63.97, 59.96, 56.65, 55.35, 55.30, 55.10, 50.30, 42.86, 42.15, 40.16, 39.68, 37.38, 36.59, 32.17, 32.13, 31.79, 31.39, 29.78, 29.13, 28.21, 24.00, 22.87, 22.77, 22.66, 20.83, 19.44, 18.33, 18.24, 14.26, 12.85, 0.10, −4.50. HR-MS (ESI): calcd for C₇₇H₁₀₂C₁₄N₂NaO₁₄Si⁺[M+Na⁺]: 1469.5752, found: 1469.5746.

ac. Synthesis of Compound 36

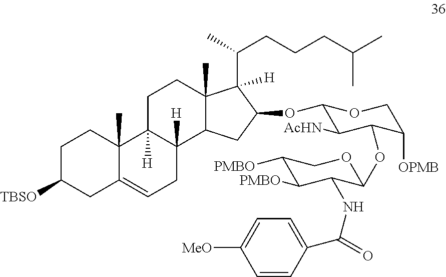

To a stirred solution of compound 35 (15 mg, 0.010 mmol) in EtOH (1 mL) was added ethylenediamine (3.5 μL, 0.0517 mmol) and stirred at 60° C. for 6 hours till the starting material was consumed. The solvent was evaporated under vaccum and the crude residue was placed under high vacuum for 2 hours. To this crude mixture, CH₂Cl₂ (1 ml) was added followed by the sequential addition of Et₃N (14 μL, 0.10 mmol) and p-anisoyl chloride (7 μL, 0.0517 mmol) at 0° C. The reaction was stirred at 0° C. for 15 minutes and warmed to room temperature and stirred for 1 hour. The reaction was quenched with saturated NaHCO₃ (2 mL), and CH₂Cl₂ (5 mL) was added to dilute the reaction mixture. The organic phase was separated and the aqueous phase was extracted with CH₂Cl₂ (10 mL×3). The combined organic phase was washed with brine and dried over Na₂SO₄. The filtrate was concentrated under vacuum and purified by column chromatography on silica gel using CH₂Cl₂—MeOH (100:1-30:1) as the eluent as the eluent to obtain the amide product 36 (10 mg) in a 70% yield for two steps. [α]_{D}¹⁹=−30.5 (c=1.0 in CHCl₃); ¹H NMR (300 MHz, CDCl₃) δ7.44-7.26 (m, 8H), 6.89-6.65 (m, 8H), 5.72 (d, J=6.2 Hz, 1H), 5.28 (d, J=2.7 Hz, 1H), 4.85-4.25 (m, 12H), 3.97 (m, 2H), 3.82 (s, 3H), 3.81 (s, 3H), 3.72 (s, 3H), 3.74 (s, 3H), 3.43 (m, 4H), 2.34-1.83 (m, 12H), 1.78-1.41 (m, 12H), 0.96 (m, 34H), 0.05 (s, 6H); ¹³C NMR (75 MHz, CDCl₃) δ170.88, 162.08, 159.61, 159.02, 154.28, 149.03, 141.61, 131.02, 130.30, 130.15, 129.53, 129.19, 128.85, 126.24, 121.28, 114.09, 113.80, 113.69, 113.59, 81.94, 80.97, 77.36, 72.82, 72.81, 72.78, 72.38, 71.07, 71.04, 69.29, 68.50, 62.83, 62.72, 60.45, 60.38, 60.36, 57.02, 55.38, 55.09, 50.37, 42.94, 42.93, 42.30, 40.08, 39.74, 37.45, 37.42, 36.69, 36.10, 36.03, 35.89, 35.80, 32.19, 31.91, 31.48, 31.31, 29.85, 29.37, 28.27, 26.09, 23.85, 22.93, 22.75, 20.92, 19.55, 18.43, 18.39, 13.13, 13.09, 7.86, −4.46; HR-MS (ESI): calcd for C₇₇H₁₁₀N₂NaO₁₄Si⁺[M+Na⁺]: 1337.7624, found: 1337.7627.

ad. Synthesis of Compound 4

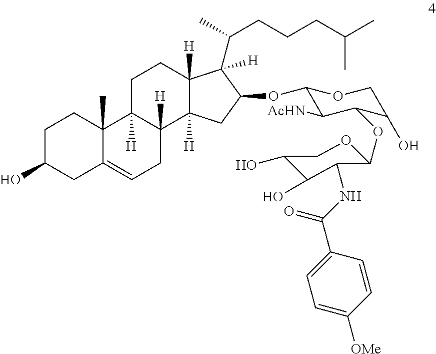

A solution of CH₂Cl₂:TFA (10:1) (2 ml) was added to compound 36 (14 mg, 0.010 mmol) at room temperature and stirred for 10 minutes. The reaction was quenched with a solution of saturated NaHCO₃ (2 mL), diluted with CH₂Cl₂ (10 mL) and dried over Na₂SO₄. The solution was concentrated under vacuum and purified by column chromatography on silica gel using CH₂Cl₂: MeOH (10:1) as the eluent to obtain the crude product 4 (6 mg) in a 67% yield. The crude product was subject to preparative TLC purification followed HPLC purification. The HPLC purification was performed using a Beckman System Gold instrument with a model 166P variable-wavelength UV detector connected to a 128 solvent module, equipped with a semi-preparative Apollo C18 column (Grace, 1.5×25 cm, 5 μm) under UV detection at 254 nm. 50-100% H₂O/MeOH solvent gradient was used to obtain the pure product 4 with a retention time of 32 minutes. [α]_{D}¹⁹=+53.33 (c=0.1 in CH₃OH); ¹H NMR (600 MHz, MeOD) δ7.92 (d, J=8.8 Hz, 1H), 6.96 (d, J=8.8 Hz, 1H), 5.31 (d, J=5.0 Hz, 1H), 4.80 (d, J=5.7 Hz, 1H), 4.68 (dd, J=12.8, 8.3 Hz, 1H), 4.11 (dd, J=15.4, 2.1 Hz, 1H), 3.97 (m, 2H), 3.94-3.91 (m, 2H), 3.89 (d, J=6.0 Hz, 1H), 3.86 (s, 1H), 3.76 (dd, J=12.0, 2.3 Hz, 1H), 3.62 (dd, J=12.0, 4.7 Hz, 1H), 3.60 -3.55 (m, 1H), 3.47 (t, J=9.3 Hz, 1H), 3.40-3.34 (m, 1H), 3.21-3.17 (m, 1H), 2.26-2.11 (m, 3H), 1.96 (s, 3H), 1.92 -1.77 (m, 4H), 1.55-0.90 (m, 30H), 0.88 (d, J=5.6 Hz, 6H), 0.72 (d, J=4.3 Hz, 3H), 0.51 (s, 3H); ¹³C NMR (151 MHz, MeOD) δ163.76, 142.23, 130.52, 127.80, 122.32, 114.69, 110.54, 102.42, 85.84, 84.36, 81.86, 76.70, 72.43, 71.71, 66.98, 64.01, 63.09, 62.00, 56.88, 56.03, 55.89, 51.63, 49.43, 49.28, 49.14, 49.00, 48.86, 48.72, 48.57, 43.14, 42.97, 41.01, 38.45, 37.64, 37.13, 33.29, 32.87, 32.76, 32.25, 31.31, 29.16, 28.34, 26.00, 23.87, 23.19, 23.16, 22.81, 21.90, 19.89, 19.15, 19.08, 14.57, 13.40; HR-MS (ESI): calcd for C₄₇H₇₁N₂O₁₁⁺[M−H⁺]: 839.5058, found: 869.5065.

### Example 2

The following illustrate representative pharmaceutical dosage forms, containing a compound of formula (I) or a pharmaceutically acceptable salt thereof (Compound X), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula (I): 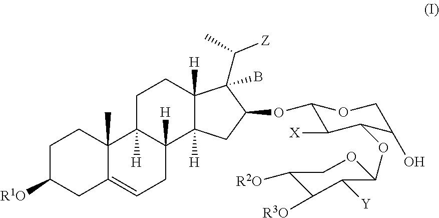
wherein:
R¹ is H, (C₁-C₆)alkyl, (C₁-C₆)alkanoyl, (C₁-C₆)alkoxycarbonyl, —C(═O)NR^{a}R^{b}, or a saccharide;
R² is H, C₁-C₆)alkyl, C₁-C₆)alkanoyl, C₁-C₆)alkoxycarbonyl, or —C(═O)NR^{a}R^{b};
R³ is H, C₁-C₆)alkyl, C₁-C₆)alkanoyl, C₁-C₆)alkoxycarbonyl, or —C(═O)NR^{a}R^{b};
B is H, hydroxy, NH₂, or C₁-C₆)alkyl;
X is hydroxy, C₁-C₆)alkoxy, C₁-C₆)alkanoyloxy, C₁-C₆)alkanoyloxycarbonyl, —O-C(═O)NR^{a}R^{b}, —NH—(C═O)OR^{c}, —NH—(C═O)NR^{c}R^{d}, —NH—S(═O)₂R^{c}, —NH—(S═O)R^{c}, —O-saccharide, cinnamoyl, or —NR^{c}R^{d};
Y is hydroxy, C₁-C₆)alkoxy, C₁-C₆)alkanoyloxy, C₁-C₆)alkanoyloxycarbonyl, —O—(═O)NR^{a}R^{b}, —NH—(C═O)OR^{c}, —NH—(C═O)NR^{c}R^{d}, —NH—S(═O)₂R^{c}, —NH—(S═O)R^{c}, —O-saccharide, cinnamoyl, or —NR^{c}R^{d};
Z is H, hydroxy, (C₁-C₈)alkyl, C₁-C₆)alkoxycarbonyl, C₁-C₆)alkanoyloxy, (C₁-C₆)alkylthiocarbonyl, C₁-C₆)alkanoylthio, (C₂-C₈)alkenyl, —C(═O)NR^{a}R^{b}, —NR^{a}R^{b}, or (C₂-C₈)alkynyl, wherein any (C₁-C₈)alkyl, C₁-C₆)alkoxycarbonyl, C₁-C₆)alkanoyloxy, (C₁-C₆)alkylthiocarbonyl, C₁-C₆)alkanoylthio, (C₂-C₈)alkenyl, and (C₂-C₈)alkynyl, is optionally substituted with one or more groups independently selected from halo and C₁-C₆)alkoxy;
each R^{a} and R^{b} is independently H, C₁-C₆)alkyl, aryl, heteroaryl, aryl(C₁-C₆)alkyl or heteroaryl(C₁-C₆)alkyl, wherein any C₁-C₆)alkyl, aryl, heteroaryl, aryl(C₁-C₆)alkyl and heteroaryl(C₁-C₆)alkyl of R^{a} and R^{b} is optionally substituted with one or more groups independently selected from halo, cyano, oxo (═O), —S(O)ₙ—R^{e}, (C₁-C₆)alkyl, (C₃-C₆)cycloalkyl, carboxy, NO₂, hydroxy, C₁-C₆)alkoxy, aryl, and heteroaryl; or R^{a} and R^{b} together with the nitrogen to which they are attached form a morpholino, piperazino, pyrrolidino or piperidino ring, wherein the morpholino, piperazino, pyrrolidino and piperidino ring is optionally substituted with one or more groups independently selected from halo, cyano, oxo (═O), (C₁-C₆)alkyl, (C₃-C₆)cycloalkyl, carboxy, NO₂, hydroxy, C₁-C₆)alkoxy, C₁-C₆)alkoxycarbonyl, and C₁-C₆)alkanoyloxy;
each R^{c} and R^{d} is independently H, C₁-C₆)alkyl, aryl, heteroaryl, aryl(C₁-C₆)alkyl or heteroaryl(C₁-C₆)alkyl, wherein any C₁-C₆)alkyl, aryl, heteroaryl, aryl(C₁-C₆)alkyl and heteroaryl(C₁-C₆)alkyl of R^{a} and R^{b} is optionally substituted with one or more groups independently selected from halo, cyano, oxo (═O), C₁-C₆)alkyl, (C₃-C₆)cycloalkyl, carboxy, NO₂, hydroxy, C₁-C₆)alkoxy, aryl, and heteroaryl; or R^{c} and R^{d} together with the nitrogen to which they are attached form a morpholino, piperazino, pyrrolidino or piperidino ring, wherein the morpholino, piperazino, pyrrolidino and piperidino ring is optionally substituted with one or more groups independently selected from halo, cyano, oxo (═O), C₁-C₆)alkyl, (C₃-C₆)cycloalkyl, carboxy, NO₂, hydroxy, C₁-C₆)alkoxy, C₁-C₆)alkoxycarbonyl, and (C₁-C₆)alkanoyloxy;
each W is independently H, C₁-C₆)alkyl, aryl, heteroaryl, aryl(C₁-C₆)alkyl or heteroaryl(C₁-C₆)alkyl, wherein any C₁-C₆)alkyl, aryl, heteroaryl, aryl(C₁-C₆)alkyl and heteroaryl(C₁-C₆)alkyl of R^{a} and R^{b} is optionally substituted with one or more groups independently selected from halo, cyano, oxo (═O), C₁-C₆)alkyl, (C₃-C₆)cycloalkyl, carboxy, NO₂, hydroxy, C₁-C₆)alkoxy, aryl, and heteroaryl; and
each n is independently 0, 1, or 2;
or a salt thereof.

2. A compound or salt as described in claim 1 wherein W is H.

3. A compound or salt as described in claim 1, wherein R² is H.

4. A compound or salt as described in claim 1, wherein R³ is H.

5. A compound or salt as described in claim 1, wherein B is H or OH.

6. A compound or salt as described in claim 1, wherein B is H.

7. A compound or salt as described in claim 1, wherein X is acetylamino.

8. A compound or salt as described in claim 1, wherein Y is 4-methoxybenzoylamino.

9. A compound or salt as described in claim 1, any one of claims 1-8 wherein Z is 4-methyl-1-pentyl.

10. A pharmaceutical composition comprising a compound of formula (I) as described in claim 1, or a pharmaceutically acceptable salt thereof, and a pharmaceutically acceptable diluent or carrier.

11. A method for treating cancer in an animal comprising administering a compound of formula (I) as described in claim 1, or a pharmaceutically acceptable salt thereof, to the animal.

12-14. (canceled)

15. A compound or salt as described in claim 1 wherein Z is (C₁-C₈)alkyl.

16. A compound or salt as described in claim 1 wherein Y is —NH—(C═O)OR^{c}, —NH—(C═O)NR^{c}R^{d}, —NH—S(═O)₂R^{c}, —NH—(S═O)R^{c}, or —NR^{c}R^{d}.

17. A compound or salt as described in claim 1 wherein Y is —NR^{c}R^{d}.

18. A compound or salt as described in claim 17 wherein each R^{a} and R^{b} is independently H, (C₁-C₆)alkyl, or aryl(C₁-C₆)alkyl, wherein any C₁-C₆)alkyl and aryl(C₁-C₆)alkyl of R^{a} and R^{b} is optionally substituted with one or more groups independently selected from halo, oxo (═O), hydroxy, and C₁-C₆)alkoxy.

19. A compound or salt as described in claim 17 wherein R^{a} is H, and R^{b} is (C₁-C₆)alkyl, or aryl(C₁-C₆)alkyl, wherein any C₁-C₆)alkyl and aryl(C₁-C₆)alkyl of R^{b} is optionally substituted with one or more groups independently selected from halo, oxo (═O), hydroxy, and C₁-C₆)alkoxy.

20. A compound or salt as described in claim 17 wherein R^{a} is H, and R^{b} is aryl(C₁-C₆)alkyl, wherein any aryl(C₁-C₆)alkyl of R^{b} is optionally substituted with one or more groups independently selected from halo, oxo (═O), hydroxy, and C₁-C₆)alkoxy.

21. A compound or salt as described in claim 17 wherein R^{a} is H, and R^{b} is aryl(C₁-C₆)alkyl, wherein any aryl(C₁-C₆)alkyl of R^{b} is optionally substituted with one or more groups independently selected from oxo (═O) and C₁-C₆)alkoxy.

22. A compound or salt as described in claim 1, which is: 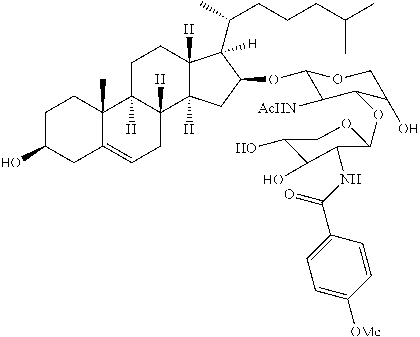
or a salt thereof.
